(19) United States
(12) Patent Application Publication
(10) Pub. No.: US 2012/0203124 A1
(43) Pub. Date: Aug. 9, 2012

(54) MOBILE PHONE FOR RECORDING ECG

(75) Inventor: Chun Leng Michael Lim, Singapore (SG)

(73) Assignee: EPHONE INTERNATIONAL PTE LTD, Singapore (SG)

(21) Appl. No.: 13/148,400

(22) PCT Filed: Sep. 29, 2009
(86) PCT No.: PCT/SG09/00358

§ 371 Date: Apr. 27, 2012

(51) Int. Cl. A61B 5/0404; H05K 13/00; A61B 5/0432

(52) U.S. Cl. 600/523; 600/509; 29/592.1

(57) ABSTRACT

A mobile phone, a method of assembling a mobile phone, and a method of recording an ECG using the mobile phone. The mobile phone comprises a casing; a wireless communication module disposed inside the casing for communicating with a mobile network; one or more sensor elements integrated on the casing for measuring an electrophysiological signal of a person; and an ECG generator module disposed inside the casing and coupled to the sensor elements and the communication module for generating an ECG from the measured electrophysiological signal and for transmitting data representing said ECG via the communication module.

### FIELD OF INVENTION

The present invention relates broadly to a mobile phone, to a method of assembling a mobile phone, and to a method of recording an ECG using the mobile phone.

### BACKGROUND

Transtelephonic monitoring systems have been used to record electrophysiological signals arising from the beating heart by placing a handheld ECG recording device, usually with 3 pins at the base, over the chest and then transmitting the data by calling a phone land line and using the ECG recording device to transmit the data over the phone land line to a remote receiving station. The ECG recording transmitted is then displayed on the monitor of the receiving station. This method of ECG transmission requires the use of a separate ECG recording device and a phone land line. One such system is described in U.S. Pat. No. 4,151,513.

Prior art Holter monitoring involves the placement of ECG electrodes over the chest of the subject and recording the electrophysiological signals of a beating heart and recording the signals in a ECG recording device attached to the electrodes by cables or wires. Prior art Holter monitoring systems have at least 3 ECG electrode pads and cables connected to a pocket device which records the data via tape or solid state hardware. Typically, the subject wears the Holter monitoring device continuously for 24 hours and the device is removed and the data is transferred to a computer with the software to display and analyse the ECG recordings. This method of monitoring is troublesome as the subject has to wear the electrodes and cables which are usually plastered down to the chest with adhesive tape and they have to constantly carry the pocket size recorder wherever they are. It is impractical to use this method of monitoring on a long term basis. One such system is described in US Patent Application Publication 2006/0025696.

Telemetry ECG monitoring involves the use of an ECG recording device which is connected to the body of the subject being monitored via ECG electrodes and cables or wires. The electrophysiological data being received by the ECG recording device is then transmitted via wireless means to a display monitor and receiving station. This is usually used in a hospital setting for patients that requires constant monitoring. Such systems are described e.g. in US Patent Application Publication 2005/0017864, US Patent Application 20050165318 and U.S. Pat. No. 7,515,044.

A modification of this technique is the use of an ECG recording device which is worn by the subject and the electrophysiological data is then transmitted to a mobile device which allows the signal to be transmitted to a remote receiving station. One such system is described in U.S. Pat. No. 7,542,878, Jun. 2, 2009.

Cardiovascular disease continues to be a major cause of mortality and morbidity. However, the above described current methods of remote ECG monitoring have limitations and are meant to be used by subjects who are suspected to have underlying disease conditions which require monitoring. The transtelephonic ECG monitoring device requires a separate ECG recording device and access to a phone land line. Holter monitoring requires the subject to constantly wear the ECG recording device and requires the ECG data to be physically downloaded into a computer. Telemetry ECG monitoring requires the subject to constantly wear the ECG recording device and while it allows the ECG recording device to continuously monitor the subject, it is generally unsuitable for ambulatory purposes. In general, these devices and systems are not meant to allow transmission of medical data across transnational boundaries.

A need therefore exists to provide an ECG recording device and associated support systems and services that seek to address at least one of the above-mentioned problems.

### SUMMARY

In accordance with a first aspect of the present invention there is provided a mobile phone comprising a casing; a wireless communication module disposed inside the casing for communicating with a mobile network; one or more sensor elements integrated on the casing for measuring an electrophysiological signal of a person; and an ECG generator module disposed inside the casing and coupled to the sensor elements and the communication module for generating an ECG from the measured electrophysiological signal and for transmitting data representing said ECG via the communication module.

Each sensor element may comprise a conductive plate disposed with a sensor surface facing outward from the casing for direct contact with a skin of the person.

Each conductive plate may comprise a contact lead extending through the casing for slide-on biased engagement with contact points disposed on a PCB inside the casing for electrically connecting each conductive plate to the ECG generator module.

The wireless communication module may transmit the data representing said ECG in a mark-up language based format.

The mark-up language based format may comprise XML.

The ECG generator module may incorporate a unique ID into the data representing the ECG.

The ECG generator may incorporate a telephone number into the data representing the ECG.

The mobile phone may further comprise an application program installed on a processor of the mobile phone for implementing a medical data user interface on the mobile phone.

The user interface may be configured for input of medical data by the user.

The user interface may be configured for incorporating the or a unique ID into said medical data for transmission via the communication device.

The ECG generator may be coupled to a screen of the mobile phone for displaying the ECG on the mobile phone.

The mobile phone may further comprise a dedicated replaceable data storage medium coupled to the ECG generator for storing data the data representing the ECG and disposed inside the casing such that the casing has to be opened for replacing the replaceable data storage medium.

The ECG generator module may comprise means for amplifying the electrophysiological signal, means for filtering the electrophysiological signal for artifacts and noise and means for converting the electrophysiological signal into digital form.

The digital format may comprise HEX data.

In accordance with a second aspect of the present invention there is provided a method of assembling a mobile phone, the method comprising the steps of providing a casing; providing a wireless communication module disposed inside the casing for communicating with a mobile network; providing one or more sensor elements integrated on the casing for measuring an electrophysiological signal of a person; and providing an ECG generator module disposed inside the casing and coupled to the sensor elements and the communication module for generating an ECG from the measured electrophysiological signal and for transmitting data representing said ECG via the communication module.

Each sensor element may comprise a conductive plate disposed with a sensor surface facing outward from the casing for direct contact with a skin of the person.

Each conductive plate may comprise a contact lead extending through the casing for slide-on biased engagement with contact points disposed on a PCB inside the casing for electrically connecting each conductive plate to the ECG generator module.

The method may further comprise configuring the wireless communication module for transmitting the data representing said ECG in a mark-up language based format.

The mark-up language based format may comprise XML.

The method may further comprise configuring the ECG generator module for incorporating a unique ID into the data representing the ECG.

The method may further comprise configuring ECG generator for incorporating a telephone number into the data representing the ECG.

The method may further comprise installing an application program on a processor of the mobile phone for implementing a medical data user interface on the method.

The user interface may be configured for input of medical data by the user.

The user interface may be configured for incorporating the or a unique ID into said medical data for transmission via the communication device.

The method may further comprise coupling the ECG generator to a screen of the mobile phone for displaying the ECG on the method.

The method may further comprise providing a dedicated replaceable data storage medium coupled to the ECG generator for storing data the data representing the ECG and disposed inside the casing such that the casing has to be opened for replacing the replaceable data storage medium.

The ECG generator module may comprise means for amplifying the electrophysiological signal, means for filtering the electrophysiological signal for artifacts and noise and means for converting the electrophysiological signal into digital form.

The digital format may comprise HEX data.

In accordance with a third aspect of the present invention there is provided a method of recording an ECG of a person, the method comprising using a mobile phone as defined in the first aspect.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1 shows a schematic diagram illustrating an ECG recording device integrated in a mobile phone device, according to an example embodiment.

FIG. 2a shows a schematic circuit diagram illustrating the components and layout of one side of a main PCB of the mobile phone device of FIG. 1.

FIG. 2b shows a schematic circuit diagram illustrating the components and layout of the other side of the main PCB of the mobile phone device of FIG. 1.

FIG. 3a shows a schematic circuit diagram of a PCB of the ECG module of the mobile phone device of FIG. 1.

FIG. 3b shows a simplified functional block diagram of the ECG module of the mobile phone device of FIG. 1.

FIGS. 4a and 4b illustrate sensors and contact points on the main PCB of the mobile phone device of FIG. 1.

FIG. 5 shows a system and process diagram of a remote wireless health monitoring system according to an example embodiment.

FIG. 6 shows a schematic diagram illustrating a backend platform of the remote wireless health monitoring system of FIG. 5.

FIG. 7 illustrates a working screen capture on a workstation of the backend platform of FIG. 6.

FIG. 8 illustrates a customer screen capture for access to a health profile via the backend platform of FIG. 6.

FIG. 9 shows a schematic drawing of a computer system for use in implementation of the backend platform of FIG. 6.

FIGS. 10a to f illustrate six different configurations for using the mobile phone device of FIG. 1 for recording ECGs according to example embodiment.

FIG. 11 shows a comparison of the morphology of the ECG recordings between the mobile phone device of FIG. 1 and a standard 12 lead ECG machine.

### DETAILED DESCRIPTION

In one aspect, the described example embodiments relate broadly to the application of a mobile phone that is able to directly record physiological data, process physiological data, display physiological data, store physiological data and transmit physiological data via telecommunication media to a remote backend server.

The example embodiments described provide a method and system for monitoring physiological data such as electrophysiological signals from a beating heart via a mobile handphone. Unlike prior art ECG monitoring devices, implementation and use of mobile phone and ECG reading device can obviate the need for the subject to wear an ECG recording device, the need to carry an additional ECG recording device and can allow the data to be transmitted across transnational boundaries. The mobile phone is a ubiquitous device that is carried by most people on a daily basis. The example embodiment can allow an ECG to be recorded instantly by direct contact of the mobile phone sensors with the body surface and the ECG pattern can be displayed on the mobile phone or sent to a remote 24 hour station. Unlike prior art ECG monitoring systems which are mainly inpatient hospital ECG monitoring systems or home care monitoring systems meant to be used by subjects who are suspected to have underlying disease conditions which require monitoring, the example embodiments provide a simple and convenient preventive system carried by subjects on a daily basis as a mobile phone and can be used as and when required whenever the need arises, and allow easy access to healthcare services as and when the need arises, with medical data being able to be recorded and transmitted to a 24 hour monitoring centre. The example embodiments can allow early detection of medical problems which can potentially be serious or life threatening.

In a further aspect, the described example embodiments relate broadly to a mobile health monitoring system that includes a mobile device which is able to perform the functions of a mobile phone and is able to record, store and transmit physiological data via wireless means to a remote monitoring and storage system. The mobile monitoring device is preferably capable of directly recording electrical signals generated by the beating heart by direct contact with the surface of the body, processing the recorded electrical signals into an ECG recording and storing the ECG recording on the mobile device or transmitting via a wireless telecommunication system to a remote monitoring and storage system.

Some portions of the description which follows are explicitly, or implicitly presented in terms of algorithms and functional or symbolic representations of operations on data within a computer memory. These algorithmic descriptions and functional or symbolic representations are the means, used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “scanning”, “calculating”, “determining”, “generating”, “initializing”, “outputting”, “displaying” or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a general purpose computer or other device selectively activated or reconfigured by a computer program stored in the computer. The algorithms and displays presented herein are not inherently related to any particular computer or other apparatus. Various general purpose machines may be used with programs in accordance with the teachings herein. Alternatively, the construction of more specialized apparatus to perform the required method steps may be appropriate. The structure of a conventional general purpose computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a general purpose computer. The computer readable medium may also include a hard-wired medium such as exemplified in the Internet system, or wireless medium such as exemplified in the GSM mobile telephone system. The computer program when loaded and executed on such a general-purpose computer effectively results in an apparatus that implements the steps of the preferred method.

The invention may also be implemented as hardware modules. More particular, in the hardware sense, a module is a functional hardware unit designed for use with other components or modules. For example, a module may be implemented using discrete electronic components, or it can form a portion of an entire electronic circuit such as an Application Specific Integrated Circuit (ASIC). Numerous other possibilities exist. Those skilled in the art will appreciate that the system can also be implemented as a combination of hardware and software modules.

A method and apparatus is disclosed to directly record physiological data, process physiological data, display physiological data, store physiological data and transmit physiological data via telecommunication media to a remote backend server.

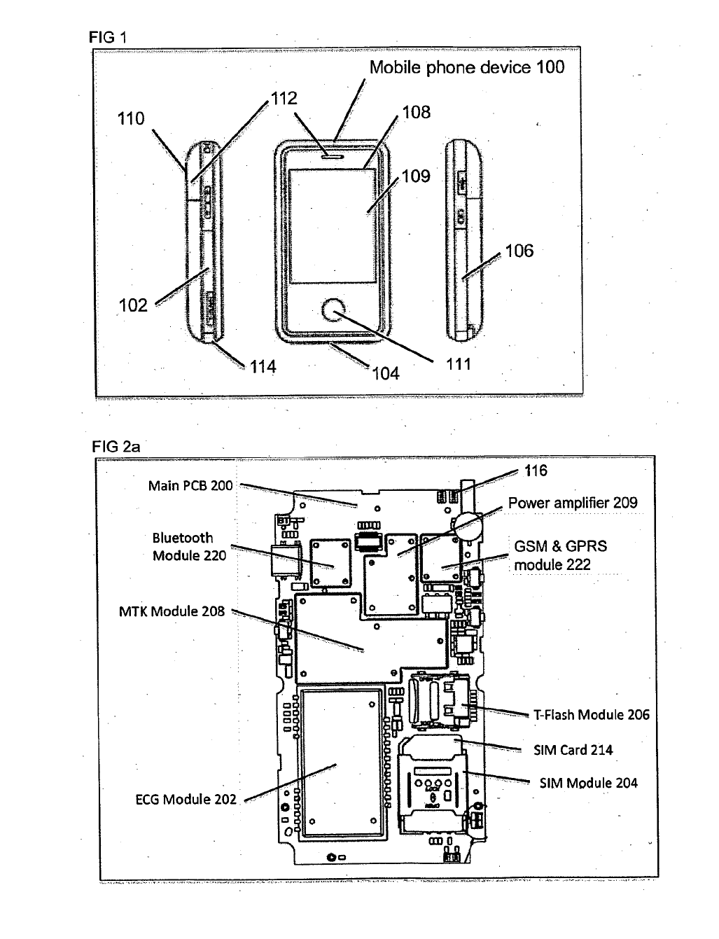

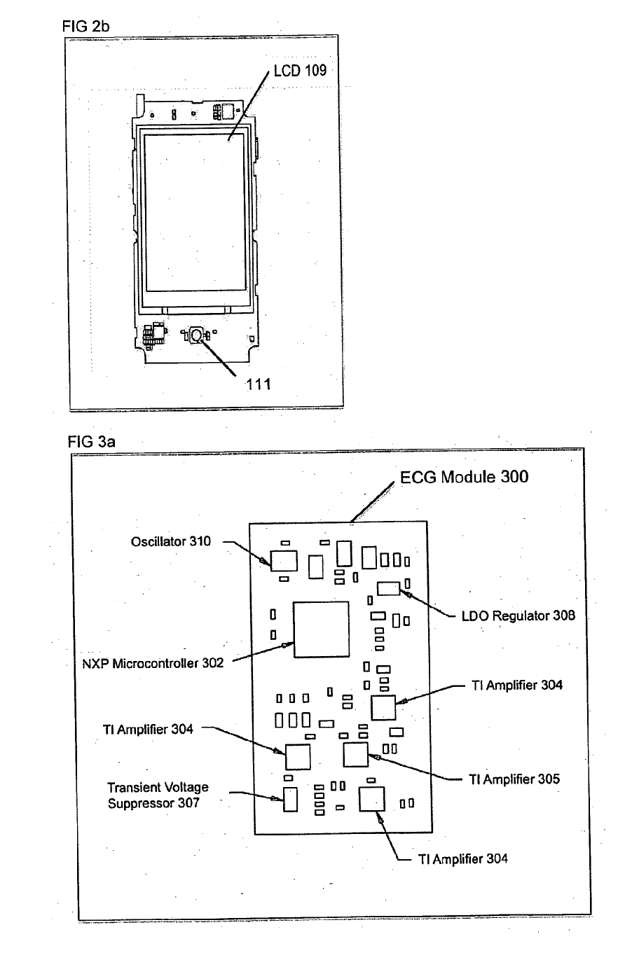

FIG. 1 illustrates an embodiment of the invention in which a mobile phone device 100 is capable of recording, inputting, processing, storing and transmitting physiological data that may include but is not limited to the following: Electrocardiogram (ECG), Blood Pressure, Heart Rate, Blood Glucose, Total Cholesterol, LDL-Cholesterol, HDL-Cholesterol and Triglycerides. In the present example embodiment, the mobile phone device 100 is adapted for measuring ECG and Heart Rate (derived from ECG), while being configured with a medical data input interface for inputting Blood Pressure, Blood Glucose, Total Cholesterol, LDL-Cholesterol, HDL-Cholesterol, Triglycerides, processing, storing and transmitting ECG, Blood Pressure, Blood Glucose, Total Cholesterol, LDL-Cholesterol, HDL-Cholesterol and Triglycerides. The mobile phone device 100 has one or more sensors e.g. conductive sensor contacts 102, 104, 106 and may include an analog amplifier, an analog comparator and an analog to digital convertor (ADC), as will be described in more detail below. The mobile phone device 100 also includes a resistive touch screen module with glass touch window 108, a LCD 109 module, a navigation button 111, a camera 110 module, a speaker 112 module, a microphone 114 module, a vibrator module, an internal antenna module 116 (FIG. 2), and a battery module.

FIGS. 2a and b illustrate the components and layout of the Main PCB 200 of the mobile phone device 100 (FIG. 1), consisting of an ECG module 202, a SIM module 204, a T-Flash memory module 206, MTK module 208, and Power amplifier module 210. In this example embodiment, the MTK module 208 used is MediaTek's MT6225 solution, which has a rich feature set including camera, support for audio and video recording and playback, e-book reader which supports .txt files, touch screen, calendar and organizer functions, Bluetooth, and GPRS capability, as are understood in the art.

The ECG module 202 records analog signals, amplifies and filters the signals before converting the signals into digital format where it is communicated to the MTK modules 208. The signal is shown real-time on the LCD 109 module (FIG. 1) of the mobile phone device 100 (FIG. 1) as it is being recorded. At the end of a recording time period, which may be, but is not limited to, 45 seconds, users are prompted on the LCD 109 screen (FIG. 1) to directly send, store or cancel the recorded ECG. The ECG module 202 is shielded by an RF shielding cover 203.

The MTK module 208 converts the data into a mark-up language based format, here Extensible Markup Language (XML) format in the example embodiment, for storage in the T-Flash memory module 206 and subsequent transmission to a backend server 604 (FIG. 6) via General Packet Radio Service (GPRS) through the wireless telecommunications network in this example embodiment. The MTK module 208 also controls the overall operation of the mobile phone device and are also shielded from interference by RF shielding cover 210.

The SIM module 204 houses a SIM card 214 for GSM networks in this example embodiment and can be opened or closed using a sliding locking mechanism. The SIM card 214 of the mobile phone device 100 (FIG. 1) enables to transmit ECG and other physiological data to a remote Internet server via GPRS over a wireless telecommunications network. The T-Flash module 206 houses an external memory card (T-Flash micro-SD format in this example embodiment), which stores all the physiological data recorded, inputted, and processed on the mobile phone device 100. The housing for the T-Flash module 206 can be opened or closed using a sliding locking mechanism. Both the SIM module 204 and the T-Flash module 206 can only be accessed by removing the back cover of the mobile phone device 100 (FIG. 1) and the battery module in this example embodiment. Additional components and functionalities of the mobile phone device can include I/O ports such as one or more USB ports, wireless links interface(s), such as e.g. Bluetooth (Bluetooth module 220), WAP, or other wireless links in addition to or as an alternative to GPRS (GSM & GPRS module 222), stylus, etc. The Power amplifier module 209 serves the purpose of amplifying the telephone signals.

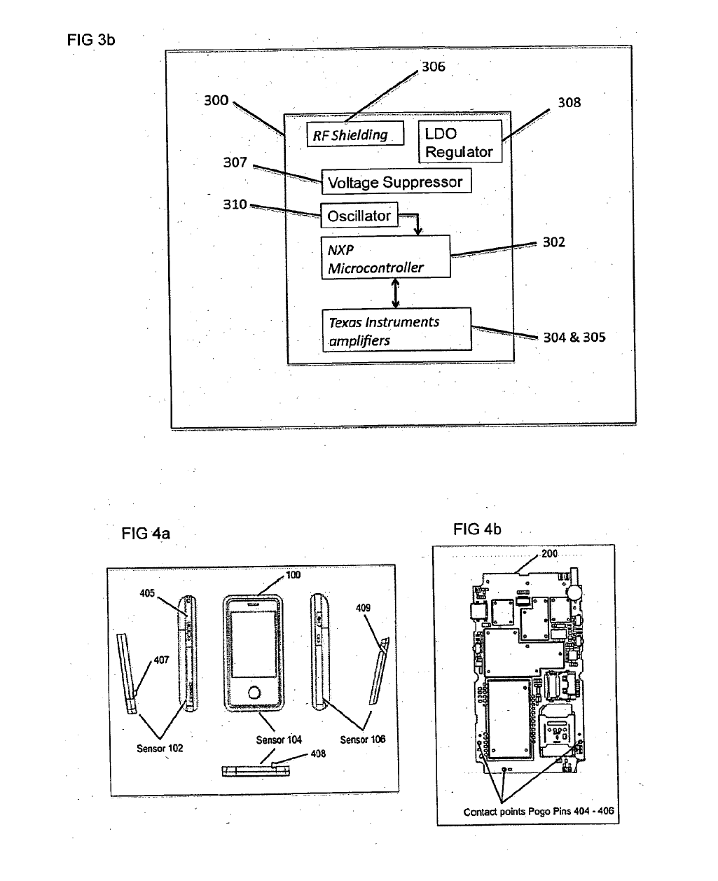

FIG. 3a shows a diagram of the PCB 300 of the ECG module 202 (FIG. 2), consisting of a NXP microcontroller chipset 302, Texas Instruments (TI) amplifier chipsets 304 and 305, oscillator 310, transient voltage suppressor 307, and LDO regulator 308, in this example embodiment.

FIG. 3b illustrates a simplified functional block diagram of the components of the ECG module 202, where the physiological data are captured in analog signals by the NXP microcontroller chipset 302, amplified by the Texas Instruments amplifier/filter chipsets 304 and 305, filtered for artifacts and noise by a configuration of capacitors and resistors, and then converted into digital form (HEX data in this example embodiment) by the NXP microcontroller chipset 302.

The RF shielding 306 serves the purpose of blocking out possible interference to the ECG module 202. The ECG module 202 in this example embodiment is programmed such that it is normally in an inactive state unless activated when the user wishes to record an ECG by pressing an icon/button on the touch screen 109 (see e.g. FIG. 1), in order to conserve battery power. The LDO regulator 308 automatically maintains a constant voltage level, the transient voltage suppressor 307 prevents voltage spikes and the oscillator generates a repeated signal as input for the NXP microcontroller chipset 302.

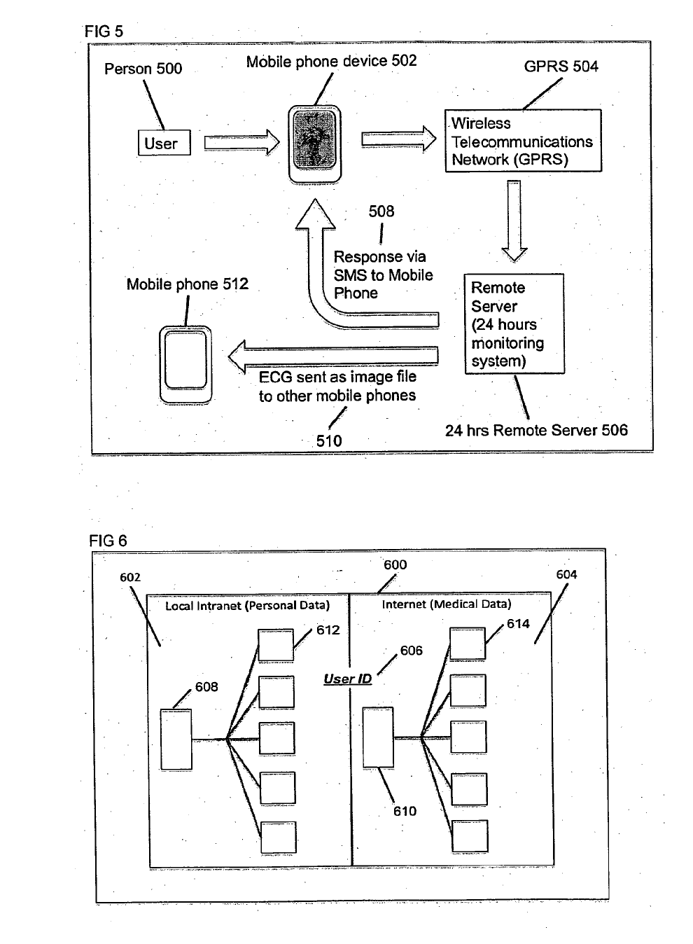

FIGS. 4a and b illustrate the sensors 102, 104, 106 and the contact points 404 to 406 on the Main PCB 200 for the purpose of obtaining the physiological data such as ECG in analog signal format through contact of the sensors 102, 104, 106 surfaces with the user. Contact points 404 to 406 make contact to contact tails 407 to 409 of the sensors 102, 104, 106 respectively when assembled.

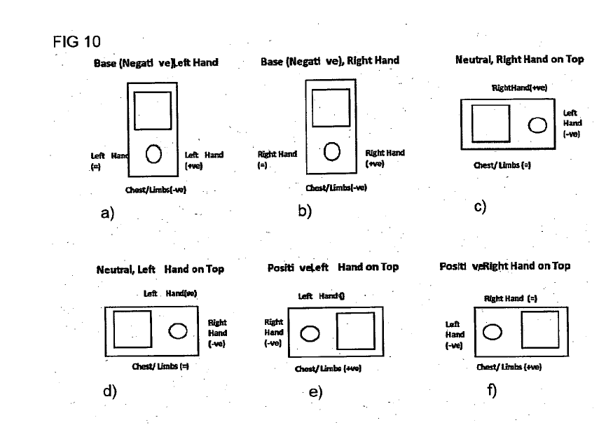

The sensors 102, 104, 106 are metallic in this example embodiment for poteritial difference measurements between different points of the body (in the case of ECG) when in contact with the user. The sensors 102, 104, 106 can be made from a variety of materials, including, but not limited to, gold, stainless steel, copper or other metals capable of enabling the detection and conduction of electro physiological signals from the sensor 102, 104, 106 surfaces to the ECG module 202, and including compound structures such as stainless steel plated with gold in one example embodiment. The sensor 102 functions as the neutral lead, the sensor 104 as the negative lead and the sensor 106 as the positive lead. Together, when the three sensors 102, 104, 106 are in contact with the user in a variety of configurations, different types of signals can be recorded in analog form. In this embodiment, the mobile phone device 100 is capable of recording all the modified pre-cordial leads (in the V1 to V6 positions, noted as mV1 to mV6) and modified limb leads I-III based on different configurations of the three sensors 102, 104, 106 in contact with the user, as will be described in more detail below with reference to FIG. 10.

As the voltage of the ECG signal is relatively low (in the range of millivolts), an amplification factor is applied to the signal obtained. Also, there is possible interference and noise from various factors such as environment and user handling, hence filtration of the signal is performed to remove or reduce the impact of these possible interferences on the integrity of the ECG signal (compare amplifier and filtration chip sets 304, 305 in FIGS. 3a and b).

The contact points 404-406 are mounted on the Main PCB 200 and push against the protruding tails 407-409 of the sensors 102, 104, 106. During assembly, the main PCB 300 is first inserted into a main housing body 405 of the mobile phone device 100, followed by assembly of the sensors 102, 104, 106 being fitted with the protruding tails 407 to 409, inserted through corresponding openings in the side-walls of the middle cover 405, configured such that the protruding tails 407 to 409 form a biased slide-on engagement with the contact points 404 to 406 respectively. In the example embodiment, double sided tape (not shown) is used to fix the sensors 102, 104, 106 in place on the middle cover 405 of the mobile phone device 100. This can advantageously help in achieving isolation of the sensors 102, 104, 106 from other components of the mobile phone device 100. However, it will be appreciated that other techniques for mounting/fixing the sensors 102, 104, 106 may be used, as would be understood in the art. The biased slide-on arrangement between the protruding tails 407 and 409 and the contact points 404 to 406 respectively can ensure direct contact and hence connection between the sensors 102, 104, 106 and Main PCB 200, thus reducing potential errors in the recording of analog signals caused by poor connection or wear and tear of wires or soldering points over time. For example, the received analog signals can be processed and converted into an ECG pattern displayed using a standard ECG grid format of speed of 25 mm/s and amplitude scale of 10 mm for 1 mV.

In this example embodiment, to carry out the ECG recordings, the mobile phone 100 is placed in six different ways in which the contact leads 102, 104, 106 come into contact with the right hand and/or left hand, and the limb or chest to obtain recordings of modified limb lead I, (mL I) modified limb lead II (mL II) and modified pre-cordial leads (mV1 to mV6 leads). FIGS. 10a to f illustrate the six different configurations.

For the modified limb leads, the bi-polar limb leads I and II are measured in this example embodiment (using the configurations in FIGS. 10a and b respectively), which according to Einthoven's Triangle, measures the electric potential between two points. Thus, for the bi-polar limb leads, there is no need for a neutral lead. However, holding the phone in one hand will typically mean that at least one finger of the hand contacts the neutral sensor (compare FIGS. 10a and b), but no measurements are processed from that sensor for the bi-polar limb leads measurements. For the modified pre-cordial leads (using the configurations in FIGS. 10c to f), the sensors are in contact with the chest, the right hand and the left hand of the user in this example embodiment. For the pre-cordial lead recordings the relevant mobile phone sensor was placed at the standard V1 to V6 positions on the chest.

To evaluate the measured ECGs obtained using the mobile phone 100 of the example embodiment, following the completion of all the mobile phone 100 ECG recordings trial persons, another ECG was then immediately recorded for each trial person using the standard 12-lead ECG machine model ESAOTE P8000, using 6 pre-cordial leads and 4 limb leads.

The modified limb and modified chest leads recorded using the mobile phone 100 in the six different positions were correlated to determine which measurement configuration best correlated with the standard 12 leads ECG. In this example embodiment, it was found that the morphology of the recorded modified limb leads of the mobile phone 100 ECG, mL I (FIG. 10a) and mL II (FIG. 10b), best correlated with lead I and lead II of the standard 12 lead ECG respectively. For the modified pre-cordial chest leads, the morphology of the ECGs recorded using the mobile phone 100 ECG in the configuration of FIG. 10e had the best correlation with the pre-cordial leads V1 to V6 of the standard 12-lead ECG.

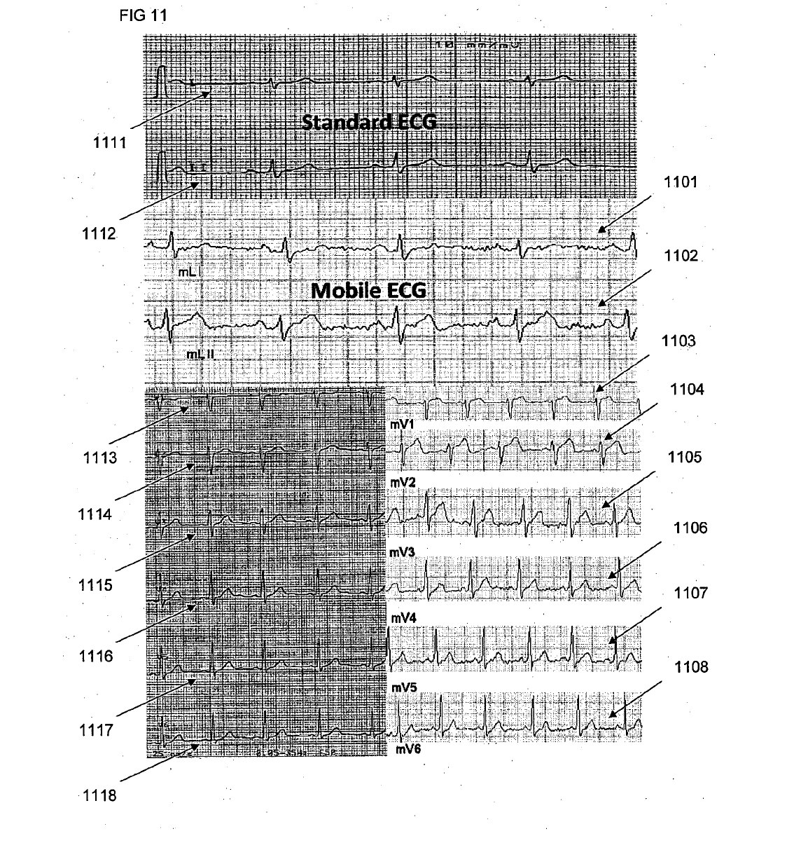

FIG. 11 shows a comparison of the morphology of the ECG recordings between the mobile phone (numerals 1101 to 1108) and the standard 12 lead ECG machine (numerals 1111 to 1118).

The morphology of the ECG recordings that were recorded using the mobile phone as compared to that of the standard 12-lead ECG recordings showed the same consistent morphological pattern (including orientation of P wave, QRS complex, ST segment and T wave) in 235 (98%) of 240 ECG trail recordings. The R wave, S wave, and R+S waves for mV1 and V1 leads were compared using a linear regression model. The amplitude measurements of the R wave, S wave and R+S wave measured in mV1 and V1 showed statistically significant correlation. For the R wave amplitude, the best fit value for the slope was 1.008 with a standard error of 0.003790 with 95% confidence intervals of 0.9782 to 0.9937. Standard deviation of residuals from line (Sy.x) was 0.01856 and the p value was <0.0001. For the S wave amplitude, the best fit value for the slope was 0.9860 with a standard error of 0.003790 with 95% confidence intervals of 0.9782 to 0.9937. Standard deviation of residuals from line (Sy.x) was 0.01764 and the p value was <0.0001. For the R+S wave amplitude, the best fit value for the slope was 0.9963 with a standard error of 0.0007529 with 95% confidence intervals of 0.9979 to 0.9948. Standard deviation of residuals from line (Sy.x) was 0.00615 and the p value was <0.0001. The PR interval and QT interval for mL II and L II leads were compared using a linear regression model. The linear regression analysis for PR Interval and QT interval for mL II and L II leads showed statistically significant correlation. For the PR interval, the best fit value for the slope was 0.9951 with a standard error of 0.000467 with 95% confidence intervals of 0.9942 to 0.9961. Standard deviation of residuals from line (Sy.x)=0.4094 and the p value was <0.0001. For the QT interval, the best fit value for the slope was 0.9125 with a standard error of 0.0001589 with 95% confidence intervals of 0.997 to 0.9984. Standard deviation of residuals from line (Sy.x)=0.4586 and the p value was <0.0001.

FIG. 5 shows a system and process diagram of a remote wireless health monitoring system 500 in one example embodiment. Person 501 uses the mobile phone device 100 to record their ECG, subsequently sending the ECG to a 24 hours monitoring system 506 via GPRS through the wireless telecommunications network 504. At the 24 hours monitoring system 506, qualified Technologists interpret the ECG and an appropriate response is sent back to person 500 e.g. via SMS 508 to their mobile phone device 502. If required, the ECG can be sent as an image file 510 such as JPEG format to any other mobile phone 512 that is capable of receiving image files, for the purpose of remote consultation between person 500 and a physician or doctor. It will be appreciated that upon receipt, the ECG image file can be viewed on the mobile phone 512 using the standard viewing tools typically provided on mobile phones capable of receiving image files, including zoom-in, zoom-out and scrolling during zooming, thus enabling a physician or doctor to view relevant portions of the ECG, notwithstanding the size of the image file.

FIG. 6 shows a schematic diagram illustrating a backend platform 600 at the 24 hours monitoring system 506 (FIG. 5) consisting of a local intranet server section 602 which hosts all personal information of the users, and an internet server section 604 which hosts all medical information of the users. When the user registers for a new user account, all the personal information such as name, age, gender, telephone number, address, etc. will be transferred and stored on the local intranet server 608, whereas all the medical information such as medical history, drug allergies, etc. will be sent and stored on the Internet server 610. The two server sections 602, 604 are segregated physically for security reasons and the only common identifier between both databases in the two servers is the user ID 606 generated by the system 600 in this example embodiment. This arrangement can preferably ensure the confidentiality of the medical information hosted on the Internet server 610 while providing the convenience and flexibility of a virtual health repository accessible from any location via the Internet, as the user ID 606 does not contain any indication of the actual identity of the user.

The local intranet server section 602 is staffed by a group of Customer Service Operators at workstations e.g. 612 who will answer to users' queries when they call in to the Service Centre hotline number. The operators will serve all requests and queries pertaining to the personal information of the users as they do not have access to any medical information of the users. The Internet server section 610 is staffed by a group of Technologists at workstation e.g. 614 who are qualified to interpret ECGs that are sent by users from their mobile phone device 100 (FIG. 5) to the Internet server 610, 24 hours a day, 7 days a week, in this example embodiment.

When the Internet server 610 receives the XML file transmitted by person 501 using their mobile phone device 100 via GPRS through the wireless telecommunications network 505 (FIG. 5), the system 600 identifies the user through the user ID 606 that is embedded in the XML data string. The XML file is then converted into an image file such as JPEG format, for easy viewing and interpretation by the Technologists at the workstations e.g. 614. In one example embodiment, the User ID is programmed into the mobile phone device 100 at the point of sale/registration during assembly and configuration, and accessible via a password-protected menu. During initial user setup, the user inputs their mobile number corresponding to the SIM card they are using in the mobile phone device 100, or any other number on which the user wishes to receive medical feedback from the system 600. Once configured on the mobile phone device 100, the backend platform 600 will then be able to ‘recognise’ the user by matching the User ID and mobile number stored in the ECG XML data stream with that stored on the backend platform 600 database. The User ID and mobile number are thus created in the database during the Registration process where the user fills in their personal particulars and medical history, if any. In this embodiment, the SIM ID, i.e. an ID number unique to the SIM that contains Mobile Country Code, Mobile Network Code and mobile station identification number, is also captured to form part of the ECG XML data stream.

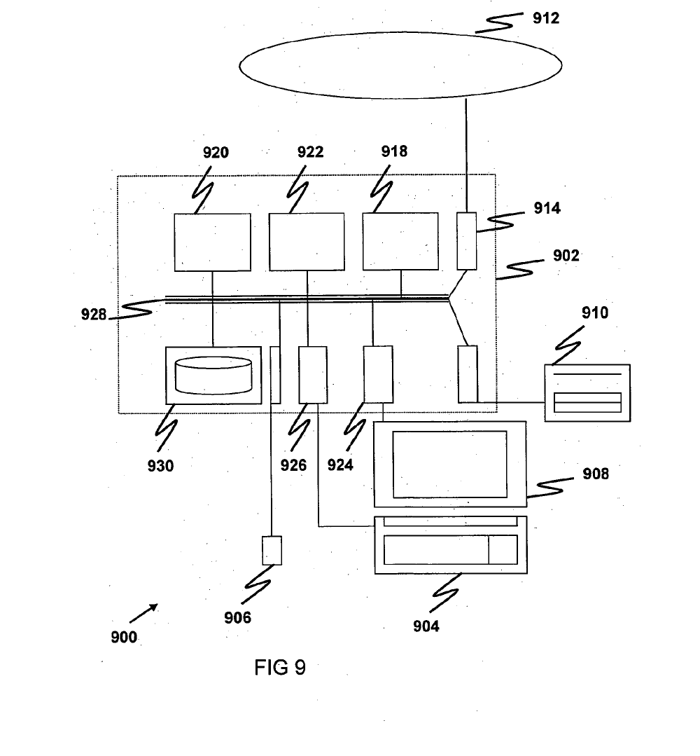

The backend platform 600 of the example embodiment can be implemented on one or more computer systems 900, schematically shown in FIG. 9. It may be implemented as software, such as a computer program being executed within the computer system 900, and instructing the computer system 900 to conduct the method of the example embodiment. The computer system 900 comprises a computer module 902, input modules such as a keyboard 904 and mouse 906 and a plurality of output devices such as a display 908, and printer 910.

The computer module 902 is connected to a computer network 912 via a suitable transceiver device 914, to enable access to e.g. the Internet (only in the case of computer systems on the Internet server section 604, FIG. 6), and other, Intranet, systems such as Local Area Network (LAN) or Wide Area Network (WAN). The computer module 902 in the example includes a processor 918, a Random Access Memory (RAM) 920 and a Read Only Memory (ROM) 922. The computer module 902 also includes a number of Input/Output (I/O) interfaces, for example I/O interface 924 to the display 908, and I/O interface 926 to the keyboard 904. The components of the computer module 902 typically communicate via an interconnected bus 928 and in a manner known to the person skilled in the relevant art.

The application program is typically supplied to the user of the computer system 900 encoded on a data storage medium such as a CD-ROM or flash memory carrier and read utilising a corresponding data storage medium drive of a data storage device 930. The application program is read and controlled in its execution by the processor 918. Intermediate storage of program data may be accomplished using RAM 920.

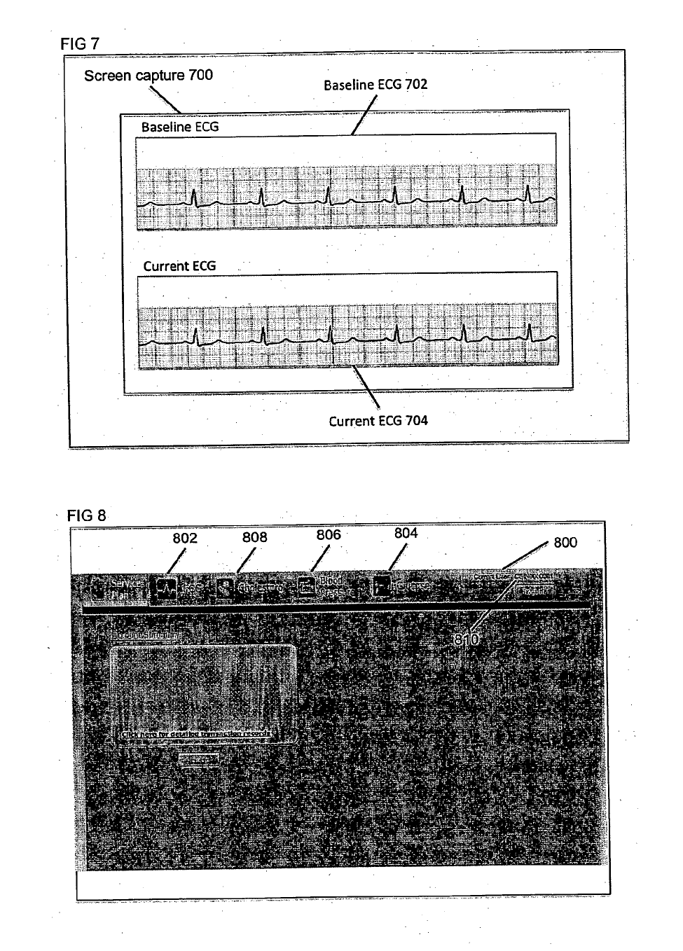

FIG. 7 illustrates a working screen capture 700 on a workstation (e.g. 610, FIG. 6) for the Technologists. A baseline ECG 702 is displayed on the top half of the screen 700 right above the current ECG 704 that is displayed on the bottom half of the screen. In this example embodiment, the ECG can be viewed on the working screen capture 700 in the standard ECG grid format of speed of 25 mm/s and amplitude scale of 10 mm for 1 mV.

The baseline ECG 702 is the first ECG that was taken by the user upon registration of a new user account with the service centre and is the basis that future ECGs will be compared against for irregularities or deviations. This arrangement advantageously allows for comparison of the current ECG 704 with the baseline ECG 702 to distinguish any irregularities or deviations, as well as careful examination of the current ECG 704, made easier by the static image format (e.g. picture) such as JPEG in this example embodiment as compared to a motion image format (e.g. video) such as MPEG.

FIG. 8 illustrates a customer screen capture 801 for access to a health profile 800 of the user after logging into their account online on the Internet. Other than the ECG log 802, health parameters such as Blood Glucose 804, Blood Pressure 806 and Cholesterol 808 are tabulated and displayed as graph trends based on user inputs transmitted from their mobile phone device 100 (FIG. 5). This allows the user to track and monitor their health parameters and health profile conveniently over the Internet, with graphical representations of the data for easy understanding and viewing over a period of time. Another use of this is for physicians or doctors to monitor the trends in the user's health parameters remotely (upon authorization by the user) and dispense appropriate advice.

As mentioned above, in the example embodiment, the health profile 800 accessible remotely through the internet server 604 (FIG. 6) does not contain any personal information of the users, but rather is associated (only) with the current user ID 810, preferably ensuring the confidentiality of the medical information while providing the convenience and flexibility of a virtual health repository accessible from any location via the internet. In other words, even if an unauthorised user such as a person performing a hacking attack on the system gains access to the health profile 800, there is no data accessible to that person that identifies the true identity and other personal information of the actual person “behind” the health profile 800. Such personal information is only kept at the intranet server 602.

A method of assembling a mobile phone according to an example embodiment comprises the steps of providing a casing; providing a wireless communication module disposed inside the casing for communicating with a mobile network; providing one or more sensor elements integrated on the casing for measuring an electrophysiological signal of a person; and providing an ECG generator module disposed inside the casing and coupled to the sensor elements and the communication module for generating an ECG from the measured electrophysiological signal and for transmitting data representing said ECG via the communication module.

### INDUSTRIAL APPLICATION

The described embodiments provide a mobile phone that is adapted to directly record physiological data such as an ECG via direct contact with the body surface preferably without the use of adhesive pads or electrodes, to process the physiological data, to display the physiological data, to store the physiological data and/or to transmit the physiological data via telecommunication media to a remote backend 24 hour monitoring centre. The data can thus also be transmitted across transnational boundaries. The data received at the 24 hour monitoring centre can be transmitted to other mobile devices, including mobile phones, and can be accessed via the internet. As an ECG can be acquired simply by direct contact between the mobile phone and transmitted by touching the touch screen of the mobile phone in the example embodiment, the simple process and the mobile phone being virtually an ubiquitous daily necessity, mean that the described embodiments can provide for convenient self monitoring and preventive healthcare assessment that can be used anywhere, anytime. Hence, anyone with chest pain, palpitations, irregular heart rhythms or symptoms suggestive of heart disease can obtain an immediate recording of his ECG and the ECG can be transmitted via the telecommunications media to the 24 hour monitoring station where access to physicians is available.

This integration of the ECG module with a mobile phone PCB module in the example embodiments advantageously enables the mobile phone device to function as both an ECG detection, recording, processing and transmitting device, and as a mobile phone.

The 24 hour monitoring and response system in example embodiments comprising of servers, computers, call centre system and a healthcare monitoring system advantageously allows medical data and parameters such as ECG, glucose, blood pressure, pulse rate, total cholesterol, LDL cholesterol, HDL cholesterol, triglycerides to be recorded and displayed. Medical parameters such as glucose, blood pressure, pulse rate, total cholesterol, LDL cholesterol, HDL cholesterol, triglycerides can be displayed both as tables and graphical format for demonstrating the trend of the parameter being monitored.

In the example embodiments, medical data is received directly from the mobile phone adapted as a detection and recording device, and is transmitted directly via existing telecommunication media and infrastructure to the 24 hour receiving and display station with no other intermediate device, and allowing the medical data to be transmitted across transnational boundaries to other countries. The servers receive the information directly from the mobile phone via the telecommunication media. While GPRS is used to transmit the medical data to the servers in one embodiment, it will be appreciated that the system can be modified to use other telecommunication media for transmission of data in other embodiments.

Medical data such as ECG can be directly transmitted from the 24 hr monitoring station to another mobile phone such as a physician's phone so that the personal or attending physician can have rapid access to the patient data. The medical data such as an ECG is sent across to the receiving mobile phone as an image file such as a jpeg file. The receiving mobile phone thus advantageously does not require special software or hardware and will be able to visualize the ECG as long as it can read image files such as jpeg files in example embodiments. This enables the patient's physician to have access to the patient's data rapidly whenever the need arises.

The personal medical data of a subject stored in the 24 hour monitoring station can be accessed by the individual person with the appropriate individualised passwords through the internet via the mobile phone or other computer device. The medical data (only) of the individual person will display and does not contain any personal data that could allow the data to be identified to be belonging to any particular individual. The medical data is tagged with an identification code. This medical data can be made available to a personal physician if the individual allows the physician to have access via the internet, e.g. by sharing the password or by setting up a secondary access account linking to the same identification code.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1-31. (canceled)

32. A mobile phone comprising:
a casing;
a wireless communication module disposed inside the casing for communicating with a mobile network;
one or more sensor elements integrated on the casing for measuring an electrophysiological signal of a person; and
an ECG generator module disposed inside the casing and coupled to the sensor elements and the communication module for converting the measured electrophysiological signal into data representing an ECG pattern for transmission of the data representing said ECG pattern via the communication module.

33. The mobile phone as claimed in claim 32, wherein each sensor element comprises a conductive plate disposed with a sensor surface facing outward from the casing for direct contact with a skin of the person.

34. The mobile phone as claimed in claim 33, wherein each conductive plate comprises a contact lead extending through the casing for slide-on biased engagement with contact points disposed on a PCB inside the casing for electrically connecting each conductive plate to the ECG generator module.

35. The mobile phone as claimed in claim 32, wherein the wireless communication module transmitts the data representing said ECG pattern in a mark-up language based format.

36. The mobile phone as claimed in claim 35, wherein the mark-up language based format comprises XML.

37. The mobile phone as claimed in claim 32, wherein the ECG generator module incorporates a unique ID into the data representing the ECG pattern.

38. The mobile phone as claimed in claim 37, wherein the ECG generator incorporates a telephone number into the data representing the ECG pattern.

39. The mobile phone as claimed in claim 32, further comprising an application program installed on a processor of the mobile phone for implementing a medical data user interface on the mobile phone.

40. The mobile phone as claimed in claim 39, wherein the user interface is configured for input of medical data by the user.

41. The mobile phone as claimed in claim 40, wherein the user interface is configured for incorporating the or a unique ID into said medical data for transmission via the communication device.

42. The mobile phone as claimed in claim 32, wherein the ECG generator is coupled to a screen of the mobile phone for displaying the ECG pattern on the mobile phone.

43. The mobile phone as claimed in claim 32, further comprising a dedicated replaceable data storage medium coupled to the ECG generator for storing the data representing the ECG pattern and disposed inside the casing such that the casing has to be opened for replacing the replaceable data storage medium.

44. The mobile phone as claimed in claim 32, wherein the ECG generator module comprises means for amplifying the electrophysiological signal, means for filtering the electrophysiological signal for artifacts and noise and means for converting the electrophysiological signal into digital form.

45. The mobile phone as claimed in claim 44, wherein the digital format comprises HEX data.

46. The mobile phone as claimed in claim 32, wherein the ECG generator module converts the measured electrophysiological signal into the ECG pattern with an ECG grid format of speed of 25 mm/s and an amplitude scale of 10 mm for 1 mV.

47. A method of assembling a mobile phone, the method comprising the steps of:
providing a casing;
providing a wireless communication module disposed inside the casing for communicating with a mobile network;
providing one or more sensor elements integrated on the casing for measuring an electrophysiological signal of a person; and
providing an ECG generator module disposed inside the casing and coupled to the sensor elements and the communication module for converting the measured electrophysiological signal into data representing an ECG pattern for transmission of the data representing said ECG pattern via the communication module.

48. The method as claimed in claim 47, wherein each sensor element comprises a conductive plate disposed with a sensor surface facing outward from the casing for direct contact with a skin of the person.

49. The method as claimed in claim 48, wherein each conductive plate comprises a contact lead extending through the casing for slid-on biased engagement with contact points disposed on a PCB inside the casing for electrically connecting each conductive plate to the ECG generator module.

50. The method as claimed in claim 47, further comprising configuring the wireless communication module for transmitting the data representing said ECG pattern in a mark-up language based format.

51. The method as claimed in claim 50, wherein the mark-up language based format comprises XML.

52. The method as claimed in claim 47, further comprising configuring the ECG generator module for incorporating a unique ID into the data representing the ECG pattern.

53. The method as claimed in claim 52, further comprising configuring ECG generator for incorporating a telephone number into the data representing the ECG pattern.

54. The method as claimed in claim 47, further comprising installing an application program on a processor of the mobile phone for implementing a medical data user interface on the method.

55. The method as claimed in claim 54, wherein the user interface is configured for input of medical data by the user.

56. The method as claimed in claim 55, wherein the user interface is configured for incorporating the or a unique ID into said medical data for transmission via the communication device.

57. The method as claimed in claim 47, further comprising coupling the ECG generator to a screen of the mobile phone for displaying the ECG pattern on the method.

58. The method as claimed in claim 47, further comprising providing a dedicated replaceable data storage medium coupled to the ECG generator for storing data the data representing the ECG pattern and disposed inside the casing such that the casing has to be opened for replacing the replaceable data storage medium.

59. The method as claimed in claim 47, wherein the ECG generator module comprises means for amplifying the electrophysiological signal, means for filtering the electrophysiological signal for artifacts and noise and means for converting the electrophysiological signal into digital form.

60. The method as claimed in claim 59, wherein the digital format comprises HEX data.

61. The mobile phone as claimed in claim 47, wherein the ECG generator module converts the measured electrophysiological signal into the ECG pattern with an ECG grid format of speed of 25 mm/s and an amplitude scale of 10 mm for 1 mV.

62. A method of recording an ECG pattern of a person, the method comprising using a mobile phone as claimed in claim 32.
